(19) United States
(12) Patent Application Publication
(10) Pub. No.: US 2025/0281229 A1
(43) Pub. Date: Sep. 11, 2025

(54) ULTRASONIC TREATMENT INSTRUMENT

(71) Applicant: OLYMPUS MEDICAL SYSTEMS CORP., Tokyo (JP)

(72) Inventors: Tsubasa NIIYAMA, Tokyo (JP); Marina Asahina, Sagamihara-shi (JP); Keisuke Nagao, Tokyo (JP); Kaito Sato, Tokyo (JP)

(21) Appl. No.: 18/984,155

(22) Filed: Dec. 17, 2024

Related U.S. Application Data
(63) Continuation of application No. PCT/JP2024/009170, filed on Mar. 8, 2024.

(51) Int. Cl. A61B 18/14; A61B 18/00

(52) U.S. Cl. CPC A61B 18/1445; A61B 2018/00589; A61B 2018/00601; A61B 2018/0063; A61B 2018/00994; A61B 2018/1452

(57) ABSTRACT

An ultrasonic treatment instrument includes: an ultrasonic blade configured to supply ultrasonic vibration and a high frequency current to a living tissue, respectively; a jaw configured to open and close with respect to the ultrasonic blade; and an abutment portion provided in the jaw and made of a first resin material, the abutment portion being configured to abut against the ultrasonic blade when the jaw is closed with respect to the ultrasonic blade, at least a part of the jaw being made of a second resin material, and the jaw including an electrode configured to supply the high frequency current on at least a part of a contact surface that is included in the jaw, the contact surface being configured to be in contact with the living tissue.

### CROSS REFERENCES TO RELATED APPLICATIONS

This application is a continuation of International Application No. PCT/JP2024/009170, filed on Mar. 8, 2024, the entire contents of which are incorporated herein by reference.

### BACKGROUND

### 1. Technical Field

The present disclosure relates to an ultrasonic treatment instrument.

### 2. Related Art

In the related art, there has been known an ultrasonic treatment instrument that supplies ultrasonic vibration and a high frequency current as treatment energy to a site (hereinafter, referred to as a treatment target) to be treated in a living tissue to treat the treatment target (see, for example, US 2021/0196334 A).

The ultrasonic treatment instrument described in US 2021/0196334 A includes an ultrasonic blade, a jaw, and an abutment portion described below.

The ultrasonic blade supplies the ultrasonic vibration and the high frequency current to the treatment target, respectively.

The jaw opens and closes with respect to the ultrasonic blade. In addition, the jaw is provided with an electrode that supplies the high frequency current to the treatment target.

The abutment portion is made of a resin material and is provided on the jaw. The abutment portion abuts against the ultrasonic blade when the jaw is closed with respect to the ultrasonic blade.

### SUMMARY

In some embodiments, an ultrasonic treatment instrument includes: an ultrasonic blade configured to supply ultrasonic vibration and a high frequency current to a living tissue, respectively; a jaw configured to open and close with respect to the ultrasonic blade; and an abutment portion provided in the jaw and made of a first resin material, the abutment portion being configured to abut against the ultrasonic blade when the jaw is closed with respect to the ultrasonic blade, at least a part of the jaw being made of a second resin material, and the jaw including an electrode configured to supply the high frequency current on at least a part of a contact surface that is included in the jaw, the contact surface being configured to be in contact with the living tissue.

The above and other features, advantages and technical and industrial significance of this invention will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to an embodiment;

FIG. 2 is a diagram for describing a configuration of a distal end portion in an ultrasonic treatment instrument;

FIG. 3 is a diagram for describing a configuration of the distal end portion in the ultrasonic treatment instrument;

FIG. 4 is a diagram for describing a configuration of a jaw;

FIG. 5 is a diagram for describing a first modification of the embodiment; and

FIG. 6 is a diagram for describing a second modification of the embodiment.

### DETAILED DESCRIPTION

Hereinafter, modes for carrying out the present invention (hereinafter, embodiments) will be described with reference to the drawings. The present invention is not limited by the embodiments described below. Furthermore, in the description of the drawings, the same portions are denoted by the same reference numerals.

### Schematic Configuration of Treatment System

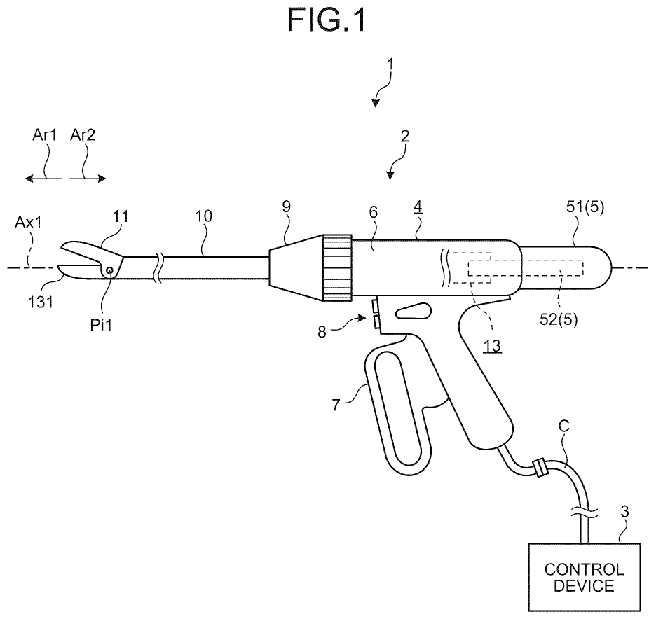

FIG. 1 is a diagram illustrating a treatment system 1 according to an embodiment.

The treatment system 1 applies treatment energy to a site to be treated (hereinafter, referred to as a treatment target) in a living tissue to treat the treatment target. The treatment energy in the present embodiment is ultrasonic energy and high frequency energy. In addition, the treatment that can be performed by the treatment system 1 according to the present embodiment is treatment such as coagulation (sealing) of the treatment target or incision of the treatment target. The coagulation and incision may be performed simultaneously. As illustrated in FIG. 1, the treatment system 1 includes a treatment instrument 2 and a control device 3.

### Configuration of Treatment Instrument

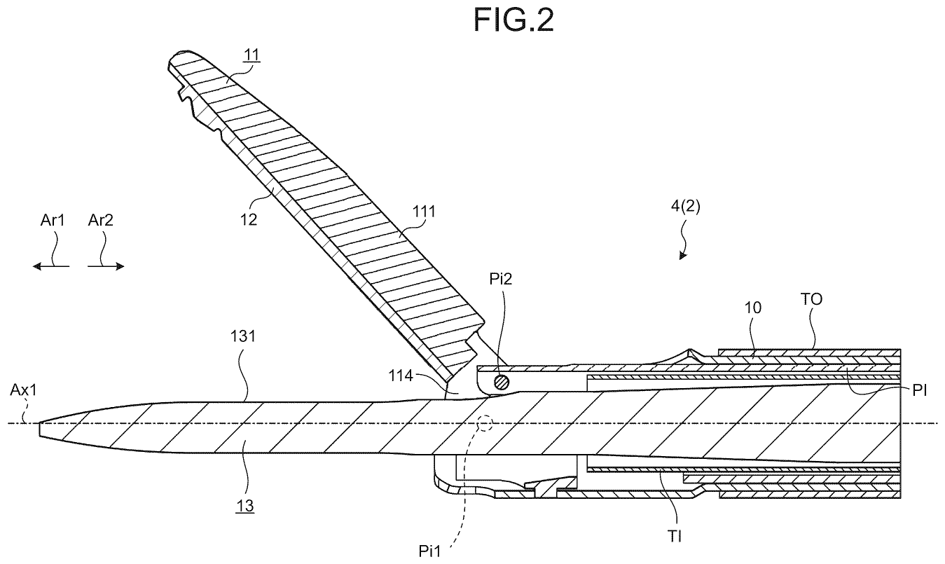

Hereinafter, one side along a central axis Ax1 (FIG. 1) of an outer pipe 10 is referred to as a distal end side Ar1, and the other side is referred to as a proximal end side Ar2. In addition, a “width direction” described below is a direction orthogonal to the central axis Ax1 and an opening/closing direction of a jaw 11 with respect to a treatment unit 131, and means a direction orthogonal to paper surfaces of FIGS. 1 and 2.

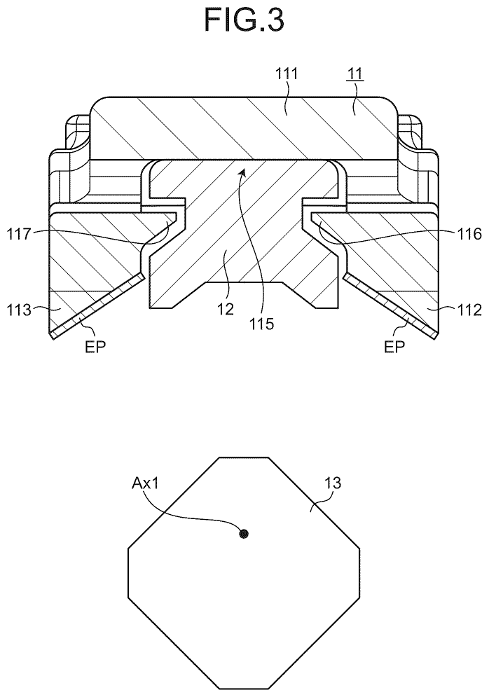

FIGS. 2 and 3 are diagrams for describing a configuration of a distal end portion in the treatment instrument 2. Specifically, FIG. 2 is a cross-sectional diagram of the distal end portion of the treatment instrument 2 cut along a plane including the central axis Ax1 of the outer pipe 10 in a state where the jaw 11 and an ultrasonic blade 13 are included in the plane. FIG. 3 is a cross-sectional diagram of the distal end portion of the treatment instrument 2 taken along a plane orthogonal to the central axis Ax1.

The treatment instrument 2 is an ultrasonic treatment instrument. Then, the treatment instrument 2 treats a treatment target by applying ultrasonic energy and high frequency energy to the treatment target. As illustrated in FIG. 1, the ultrasonic treatment instrument 2 includes a handpiece 4 and an ultrasonic transducer portion 5.

As illustrated in FIGS. 1 to 3, the handpiece 4 includes a fixed handle 6 (FIG. 1), an operation handle 7 (FIG. 1), a switch 8 (FIG. 1), a rotary knob 9 (FIG. 1), the outer pipe 10 (FIGS. 1 and 2), the jaw 11, an abutment portion 12 (FIGS. 2 and 3), and the ultrasonic blade 13.

The fixed handle 6 is a portion that supports the entire treatment instrument 2 and is gripped by an operator (user) such as a surgeon.

The operation handle 7 is movably attached to the fixed handle 6 and receives an opening/closing operation by the operator such as a surgeon.

The switch 8 is provided in a state of being exposed to an outside of the fixed handle 6, and receives a treatment operation by the operator such as a surgeon.

The rotary knob 9 has a substantially cylindrical shape coaxial with the central axis Ax1, and is provided on the distal end side Ar1 of the fixed handle 6. Then, the rotary knob 9 receives a rotation operation by the operator such as a surgeon. By the rotation operation, the rotary knob 9 rotates about the central axis Ax1 with respect to the fixed handle 6. The outer pipe 10, the jaw 11, the abutment portion 12, and the ultrasonic blade 13 rotate about the central axis Ax1 by the rotation of the rotary knob 9.

The outer pipe 10 has a tubular shape and corresponds to a pipe. In the present embodiment, the outer pipe 10 is a cylindrical pipe made of an electrically conductive material such as metal.

In the outer pipe 10, a first pin Pi1 (FIGS. 1 and 2) having a cylindrical shape extending in a direction orthogonal to the paper surfaces of FIGS. 1 and 2 and engaging with the jaw 11 and pivotally supporting the jaw 11 in a rotatable manner is fixed to an end portion on the distal end side Ar1. In the present embodiment, the first pin Pi1 is made of an electrically conductive material such as metal. The first pin Pi1 corresponds to a pin.

An outer peripheral surface of the outer pipe 10 is covered with an electrically insulating outer tube TO (FIG. 2). Further, a tubular inner pipe PI (FIG. 2) that moves forward and backward along a longitudinal direction of the outer pipe 10 according to the opening/closing operation to the operation handle 7 by the operator such as a surgeon is inserted into the outer pipe 10. A second pin Pi2 (FIG. 2) having a cylindrical shape extending in a direction orthogonal to the paper surfaces of FIGS. 1 and 2 and engaging with the jaw 11 is fixed to an end portion of the inner pipe PI on the distal end side Ar1. In the present embodiment, the second pin Pi2 is disposed on an upper side (a side on which a jaw main body 111 is disposed with respect to the treatment unit 131) in FIG. 2 with respect to the first pin Pi1.

The jaw 11 is coupled to the outer pipe 10 by the first pin Pi1. The jaw 11 is coupled to the inner pipe PI by the second pin Pi2. Then, the jaw 11 rotates about the first pin Pi1 with respect to the outer pipe 10 in conjunction with the forward and backward movement of the inner pipe PI according to the opening/closing operation to the operation handle 7 by the operator such as a surgeon. As a result, the jaw 11 is opened and closed with respect to the treatment unit 131 which is the end portion of the ultrasonic blade 13 on the distal end side, and can grip a treatment target with the treatment unit 131 therebetween.

Note that the treatment instrument 2 may be a push-close type or a pull-close type.

The push-close type has the following configuration.

The jaw 11 rotates about the first pin Pi1 in a direction approaching the treatment unit 131 in conjunction with the movement of the inner pipe PI toward the distal end side Ar1. That is, the jaw 11 is closed with respect to the treatment unit 131. The jaw 11 rotates about the first pin Pi1 in a direction away from the treatment unit 131 in conjunction with the movement of the inner pipe PI toward the proximal end side Ar2 . . . . That is, the jaw 11 opens with respect to the treatment unit 131.

The pull-close type has the following configuration.

The jaw 11 rotates about the first pin Pi1 in the direction approaching the treatment unit 131 in conjunction with the movement of the inner pipe PI toward the proximal end side Ar2. That is, the jaw 11 is closed with respect to the treatment unit 131. The jaw 11 rotates about the first pin Pi1 in a direction away from the treatment unit 131 in conjunction with the movement of the inner pipe PI toward the distal end side Ar1. That is, the jaw 11 opens with respect to the treatment unit 131.

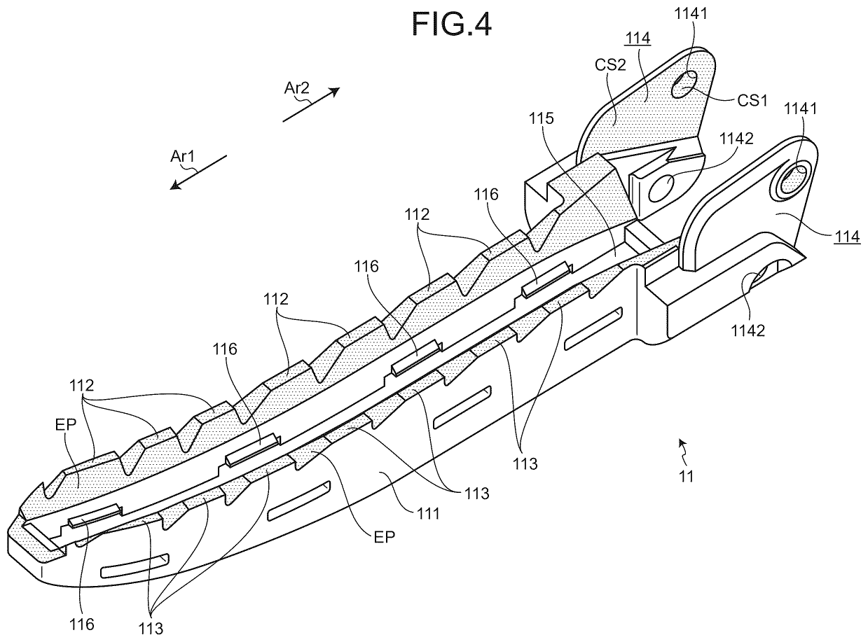

The jaw 11 is provided with an electrode EP and first and second electrically conductive surfaces CS1 and CS2 (see FIG. 4).

A detailed configuration of the jaw 11 will be described in “Configuration of Jaw” described later. The configurations of the electrode EP and the first and second electrically conductive surfaces CS1 and CS2 will be described in “Configurations of Electrode and First and Second Electrically conductive Surfaces” described later.

The abutment portion 12 is made of a first resin material having electrical insulation properties and biocompatibility, for example, polytetrafluoroethylene (PTFE), and has a substantially rectangular parallelepiped shape extending along the longitudinal direction of the jaw 11. Then, as illustrated in FIGS. 2 and 3, the abutment portion 12 is fixed to the surface of the jaw main body 111 on a side of the treatment unit 131, and abuts against the treatment unit 131 when the jaw 11 is closed with respect to the treatment unit 131. The abutment portion 12 has a function of preventing the treatment unit 131 which is ultrasonically vibrated from being damaged by colliding with the jaw 11 when the incision of the treatment target by ultrasonic vibration is completed.

The ultrasonic blade 13 is made of an electrically conductive material and has an elongated shape extending along the central axis Ax1. In addition, as illustrated in FIG. 2, the ultrasonic blade 13 is inserted into the inner pipe PI in a state where the treatment unit 131 protrudes to the outside. At this time, an end portion of the ultrasonic blade 13 on the proximal end side Ar2 is mechanically coupled to an ultrasonic transducer 52 configuring the ultrasonic transducer portion 5 as illustrated in FIG. 1. Then, the ultrasonic blade 13 transmits ultrasonic vibration generated by the ultrasonic transducer portion 5 from the end portion on the proximal end side Ar2 to the treatment unit 131. The ultrasonic vibration is longitudinal vibration that vibrates in a direction along the central axis Ax1. In the ultrasonic blade 13, an outer peripheral surface excluding the treatment unit 131 is covered with an electrically insulating inner tube TI (FIG. 2).

As illustrated in FIG. 1, the ultrasonic transducer portion 5 includes a transducer (TD) case 51 and the ultrasonic transducer 52.

The TD case 51 supports the ultrasonic transducer 52 and is detachably coupled to the fixed handle 6.

The ultrasonic transducer 52 generates ultrasonic vibration under the control of the control device 3. In the present embodiment, the ultrasonic transducer 52 is configured by a bolted Langevin transducer (BLT).

### Configuration of Control Device

The control device 3 integrally controls an operation of the treatment instrument 2 via an electric cable C (FIG. 1).

Specifically, the control device 3 detects a treatment operation on the switch 8 by the operator such as a surgeon via the electric cable C. Then, when detecting the treatment operation, the control device 3 applies treatment energy to the treatment target gripped between the jaw 11 and the treatment unit 131 via the electric cable C. That is, the control device 3 treats the treatment target.

For example, when applying ultrasonic energy to the treatment target, the control device 3 supplies drive power to the ultrasonic transducer 52 via the electric cable C. As a result, the ultrasonic transducer 52 generates longitudinal vibration (ultrasonic vibration) that vibrates in a direction along the central axis Ax1. In addition, the treatment unit 131 vibrates with a desired amplitude by the longitudinal vibration. Then, the ultrasonic vibration is supplied from the treatment unit 131 to the treatment target gripped between the jaw 11 and the treatment unit 131. The ultrasonic energy is applied from the treatment unit 131 to the treatment target.

Furthermore, for example, when applying high frequency energy to the treatment target, the control device 3 supplies high frequency power between the electrode EP provided on the jaw 11 and the ultrasonic blade 13 via the electric cable C or the like. When the high frequency power is supplied between the electrode EP and the ultrasonic blade 13, a high frequency current is supplied to the treatment target gripped between the jaw 11 and the treatment unit 131. In other words, the high frequency energy is applied to the treatment target.

### Configuration of Jaw

FIG. 4 is a diagram for describing a configuration of the jaw 11. Specifically, FIG. 4 is a perspective diagram of the jaw 11 as viewed from the side of the treatment unit 131. In FIG. 4, dots are added to the electrode EP and the first and second electrically conductive surfaces CS1 and CS2 for convenience of description.

The jaw 11 is made of a second resin material having electrical insulation properties and biocompatibility, for example, polyether ether ketone (PEEK) or polyphenyl sulfone (PPSU). As illustrated in FIG. 4, the jaw 11 is a member in which the jaw main body 111, a plurality of first tooth portions 112, a plurality of second tooth portions 113, and a pair of bearing portions 114 are integrally formed.

The jaw main body 111 is formed of an elongated plate body, and one plate surface is disposed in a posture facing the ultrasonic blade 13.

As illustrated in FIG. 4, the plurality of first tooth portions 112 respectively protrude from one side in the width direction of the surface of the jaw main body 111 on the side of the treatment unit 131 toward the side of the treatment unit 131, and are arranged side by side along the longitudinal direction of the jaw main body 111.

As illustrated in FIG. 4, the plurality of second tooth portions 113 respectively protrude from the other side in the width direction of the surface of the jaw main body 111 on the side of the treatment unit 131 toward the side of the treatment unit 131, and are arranged side by side along the longitudinal direction of the jaw main body 111.

Here, on the surface of the jaw main body 111 on the side of the treatment unit 131, at the center portion in the width direction located between the plurality of first tooth portions 112 and the plurality of second tooth portions 113, as illustrated in FIGS. 3 and 4, a recess 115 that is recessed on a side separated from the treatment unit 131 and extending along the longitudinal direction of the jaw main body 111 is provided. Further, claw portions 116 and 117 (FIGS. 3 and 4) protruding toward the center in the width direction and extending along the longitudinal direction of the jaw main body 111 are respectively provided in side wall portions on both sides in the width direction of the jaw main body 111 configuring the recess 115. Then, the abutment portion 12 is mechanically fixed to the jaw 11 by being locked to the claw portions 116 and 117. That is, the abutment portion 12 is provided at the center portion of the jaw 11 in the width direction.

The pair of bearing portions 114 are provided at end portions of the jaw main body 111 on the proximal end side Ar2, and are configured by plate bodies facing each other in the width direction of the jaw main body 111. The pair of bearing portions 114 have the same configuration. Therefore, only the configuration of one bearing portion 114 will be described below.

As illustrated in FIG. 4, the bearing portion 114 is provided with first and second insertion holes 1141 and 1142 penetrating the front and back, respectively. The bearing portion 114 is coupled to the outer pipe 10 by inserting the first pin Pi1 into the first insertion hole 1141. That is, the first insertion hole 1141 corresponds to an insertion hole. The bearing portion 114 is coupled to the inner pipe PI by inserting the second pin Pi2 into the second insertion hole 1142.

Configurations of Electrode and First and Second Electrically conductive Surfaces

As illustrated in FIG. 4, the electrode EP is provided on each of inner surfaces of the plurality of first tooth portions 112 in the width direction (on the side of second tooth portion 113), distal end surfaces of the first tooth portions 112, inner surfaces of the plurality of second tooth portions 113 in the width direction (on the side of first tooth portion 112), and distal end surfaces of the second tooth portions 113. In addition, a coating material having non-adhesiveness to the treatment target is applied onto the electrode EP. The coating material is an extremely thin coating material including fluorine or silicon and having a thickness of about several hundred nm to several μm.

As illustrated in FIG. 4, the first electrically conductive surface CS1 is provided on an inner surface of the first insertion hole 1141.

As illustrated in FIG. 4, the second electrically conductive surface CS2 is provided on an inner surface of the bearing portion 114 in the width direction (on the side of the other bearing portion 114), and electrically couples the electrode EP and the first electrically conductive surface CS1.

The electrode EP and the first and second electrically conductive surfaces CS1 and CS2 described above are formed by the following three-dimensional plating processing.

Specifically, the jaw 11 made of the second resin material is irradiated with a laser at a predetermined position. Thereafter, the electrode EP and the first and second electrically conductive surfaces CS1 and CS2 are formed at the laser irradiation position by electroless plating. A thickness of the electrode EP and the first and second electrically conductive surfaces CS1 and CS2 is, for example, about several μm. In addition, a formed product produced by such three-dimensional plating processing is called a molded interconnect device (MID) as a product in which an electrode circuit is formed on the surface of a three-dimensional resin molded article. The electrode EP and the first and second electrically conductive surfaces CS1 and CS2 are not limited to those formed by the above-described three-dimensional plating processing, and those formed by other methods may be adopted.

Then, when high frequency power is supplied to the electrode EP in order to apply high frequency energy to the treatment target, the high frequency power is supplied following an electric path of the electric cable C to the outer pipe 10 to the first pin Pi1 to the first electrically conductive surface CS1 to the second electrically conductive surface CS2 and to the electrode EP.

According to the present embodiment described above, the following effects are obtained.

In the treatment instrument 2 according to the present embodiment, at least a part of the jaw 11 is made of the second resin material. The jaw 11 is provided with the electrode EP described above. Therefore, rigidity of the jaw 11 can be made smaller than that in a case where it is made of a metal material. That is, even when the abutment portion 12 is worn by repeatedly outputting ultrasonic vibration, a distance between the ultrasonic blade 13 and the jaw 11 decreases, and the ultrasonic blade 13 and the jaw 11 come into contact with each other, an influence on the ultrasonic blade 13 can be reduced.

Therefore, the treatment instrument 2 according to the present embodiment can reduce an influence on a life of the ultrasonic blade 13.

In particular, the electrode EP is formed by three-dimensional plating processing. Therefore, even when the jaw 11 is made of the second resin material, the electrode EP can be easily formed at a specific position in the jaw 11.

In addition, a coating material having non-adhesiveness to the treatment target is provided on the electrode EP. Therefore, it is possible to prevent the treatment target from adhering to the electrode EP and to favorably treat the treatment target.

### OTHER EMBODIMENTS

Although the embodiment for carrying out the present invention have been described so far, the present invention should not be limited only by the above-described embodiment.

In the above-described embodiment, the outer pipe 10 is adopted as the electric path from the electric cable C to the electrode EP, but the disclosure is not limited thereto, and the inner pipe PI may be adopted instead of the outer pipe 10. That is, the pipe is not limited to the outer pipe 10, and may be the inner pipe PI.

In the above-described embodiment, the following first and second modifications may be adopted.

### First Modification

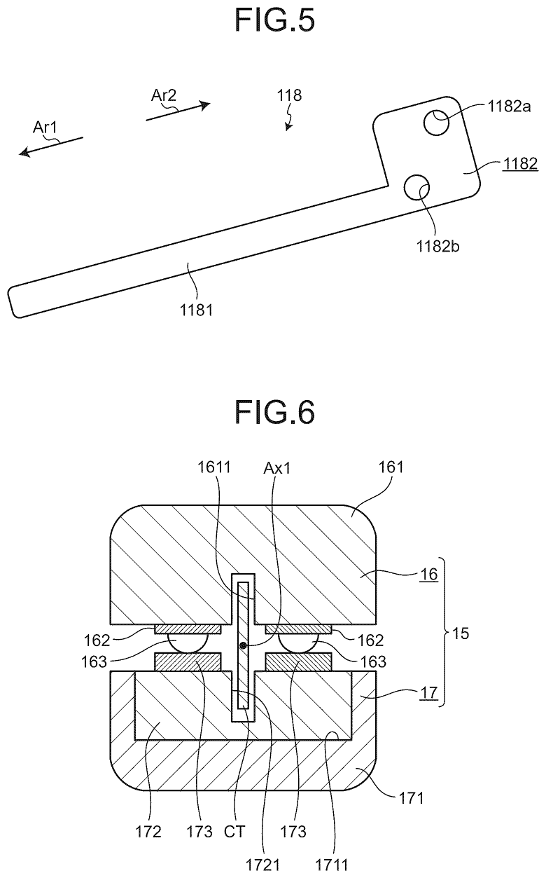

FIG. 5 is a diagram for describing a first modification of the embodiment. Specifically, FIG. 5 is a diagram illustrating a metal portion 118 provided inside the jaw 11.

In the above-described embodiment, as in the present first modification illustrated in FIG. 5, the metal portion 118 for securing rigidity of the jaw 11 may be provided inside the jaw 11.

As illustrated in FIG. 5, the metal portion 118 includes a metal portion main body 1181 serving as a base of the jaw main body 111, the plurality of first tooth portions 112, and the plurality of second tooth portions 113, and a metal portion bearing portion 1182 serving as a base of the pair of bearing portions 114.

Here, as illustrated in FIG. 5, the metal portion bearing portion 1182 is provided with a first insertion hole 1182a corresponding to the first insertion hole 1141 and a second insertion hole 1182b corresponding to the second insertion hole 1142.

Then, the jaw 11 is formed by molding (insert molding) or coating the second resin material on an outer peripheral surface of the metal portion 118. The electrode EP is formed on an outer surface of the jaw 11 by the three-dimensional plating processing described in the above-described embodiment. The electrode EP is not limited to one formed by the above-described three-dimensional plating processing, and one formed by another method may be adopted.

According to the first modification described above, the following effects are obtained in addition to the same effects as those of the above-described embodiment.

In the treatment instrument 2 according to the above-described embodiment, the metal portion 118 is provided inside the jaw 11. Therefore, the rigidity of the jaw 11 can be secured, and the life of the jaw 11 can be extended.

### Second Modification

FIG. 6 is a diagram for describing a second modification of the embodiment. Specifically, FIG. 6 is a cross-sectional diagram corresponding to FIG. 3, and is a cross-sectional diagram in which a distal end portion of the treatment instrument 2 according to the second modification is cut by a plane orthogonal to the central axis Ax1.

In the above-described embodiment, ultrasonic energy and high frequency energy are adopted as treatment energy applied to a treatment target by the treatment instrument 2, but the disclosure is not limited thereto, and only high frequency energy may be used.

In the treatment instrument 2 according to the present second modification, as illustrated in FIG. 6, a gripping portion 15 is adopted instead of the jaw 11, the abutment portion 12, and the ultrasonic blade 13.

The gripping portion 15 is a portion that grips a treatment target and treats the treatment target by applying high frequency energy to the treatment target. As illustrated in FIG. 6, the gripping portion 15 includes first and second gripping portions 16 and 17.

As illustrated in FIG. 6, the first gripping portion 16 includes a first jaw 161, a first electrode 162, and an abutment portion 163.

The first jaw 161 is formed in an elongated shape extending along the central axis Ax1. The end portions of the first jaw 161 on the proximal end side Ar2 are coupled to the outer pipe 10 and the inner pipe PI by the first and second pins Pi1 and Pi2, respectively, similarly to the above-described embodiment. The first jaw 161 opens and closes with respect to the second gripping portion 17 similarly to the jaw 11 described in the above-described embodiment.

In the first jaw 161, as illustrated in FIG. 6, a surface on the side of the second gripping portion 17 is provided with a cutter groove portion 1611 located at the center in the width direction and extending from the proximal end of the first jaw 161 toward the distal end side Ar1 along the central axis Ax1.

The first jaw 161 described above is made of a resin material having electrical insulation properties and biocompatibility, for example, polyether ether ketone (PEEK) or polyphenyl sulfone (PPSU), similarly to the jaw 11 described in the above-described embodiment.

The first electrode 162 is a portion to which high frequency power is supplied from the control device 3 between a second electrode 173 (FIG. 6) configuring the second gripping portion 17. The first electrode 162 has a U-shape planarly surrounding the cutter groove portion 1611, and is provided on a surface of the first jaw 161 on the side of the second gripping portion 17 in a posture in which both ends of the U-shape face the proximal end side Ar2.

The first electrode 162 described above is formed by three-dimensional plating processing similarly to the electrode EP described in the above-described embodiment. The electrode EP is not limited to one formed by the above-described three-dimensional plating processing, and one formed by another method may be adopted.

In addition, a coating material having non-adhesiveness to the treatment target is applied onto the first electrode 162. The coating material is an extremely thin coating material including fluorine or silicon and having a thickness of about several hundred nm to several μm.

The abutment portion 163 has a hemispherical shape made of an electrically insulating material, and is provided on a surface of the first electrode 162 on the side of the second gripping portion 17. Then, the abutment portion 163 abuts on the second electrode 173 when the first gripping portion 16 is closed with respect to the second gripping portion 17. That is, the abutment portion 163 prevents the first and second electrodes 162 and 173 from being short-circuited to each other.

As illustrated in FIG. 6, the second gripping portion 17 includes a second jaw 171, a support portion 172, and the second electrode 173.

The second jaw 171 is a portion in which a part of the outer pipe 10 extends to the distal end side Ar1, and is formed in an elongated shape extending along the central axis Ax1.

In the second jaw 171, as illustrated in FIG. 6, a surface on the side of the first gripping portion 16 is provided with a recess 1711 located at the center in the width direction and extending along the central axis Ax1.

The support portion 172 is an elongated flat plate extending along the central axis Ax1, and has an outer shape substantially the same as an inner shape of the recess 1711. The support portion 172 is fitted in the recess 1711. The support portion 172 is made of, for example, an electrically insulating material having a low thermal conductivity such as PEEK. The support portion 172 is disposed between the second electrode 173 and the second jaw 171. That is, by providing the support portion 172, the second jaw 171 and the second electrode 173 are electrically insulated.

In the support portion 172, as illustrated in FIG. 6, a cutter groove portion 1721 extending from the proximal end toward the distal end side Ar1 along the central axis Ax1 is provided in the center portion of the surface on the side of the first gripping portion 16 in the width direction. The cutter groove portion 1721 faces the cutter groove portion 1611 when the first gripping portion 16 is closed with respect to the second gripping portion 17.

The second electrode 173 is a portion to which high frequency power is supplied from the control device 3 between the first electrode 162. The second electrode 173 is made of an electrically conductive material such as copper, and is a flat plate having a U-shape that planarly surrounds the cutter groove portion 1721. Then, the second electrode 173 is fixed to the surface of the support portion 172 on the side of the first gripping portion 16 in a posture in which both ends of the U-shape face the proximal end side Ar2.

In addition, a coating material having non-adhesiveness to the treatment target is applied onto the second electrode 173. The coating material is an extremely thin coating material including fluorine or silicon and having a thickness of about several hundred nm to several μm.

In addition, as illustrated in FIG. 6, the gripping portion 15 is provided with a cutter CT which is located in the cutter groove portions 1611 and 1721 and moves forward and backward along the central axis Ax1 in accordance with an operation of an operation lever (not illustrated) by the operator. That is, a target site gripped between the first and second gripping portions 16 and 17 is incised by the forward and backward movement of the cutter CT.

According to an ultrasonic treatment instrument of the disclosure, an influence on a life of an ultrasonic blade can be reduced.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic treatment instrument comprising:
an ultrasonic blade configured to supply ultrasonic vibration and a high frequency current to a living tissue, respectively;
a jaw configured to open and close with respect to the ultrasonic blade; and
an abutment portion provided in the jaw and made of a first resin material, the abutment portion being configured to abut against the ultrasonic blade when the jaw is closed with respect to the ultrasonic blade,
at least a part of the jaw being made of a second resin material, and
the jaw including an electrode configured to supply the high frequency current on at least a part of a contact surface that is included in the jaw, the contact surface being configured to be in contact with the living tissue.

2. The ultrasonic treatment instrument according to claim 1, wherein
the abutment portion is provided at a center portion of the jaw in a width direction, and
the electrode is provided on at least a part of each of contact surfaces located on both sides of the abutment portion in the width direction.

3. The ultrasonic treatment instrument according to claim 1, wherein the second resin material is polyether ether ketone or polyphenyl sulfone.

4. The ultrasonic treatment instrument according to claim 1, wherein the electrode is formed by three-dimensional plating processing.

5. The ultrasonic treatment instrument according to claim 1, further comprising:
a cylindrical pin; and
a pipe to which the pin is attached, the pipe rotatably supporting the jaw by the pin, wherein
the electrode is electrically coupled to the pipe via the pin.

6. The ultrasonic treatment instrument according to claim 5, wherein
the jaw is provided with an insertion hole through which the pin is inserted, and
an inner surface of the insertion hole is provided with a first electrically conductive surface configured to electrically couple the pin and the electrode.

7. The ultrasonic treatment instrument according to claim 6, wherein
the jaw is provided with a second electrically conductive surface configured to electrically couple the first electrically conductive surface and the electrode.

8. The ultrasonic treatment instrument according to claim 1, wherein a coating material having non-adhesiveness to the living tissue is provided on the electrode.

9. The ultrasonic treatment instrument according to claim 1, wherein
a metal portion for securing rigidity of the jaw is provided inside the jaw.
